(19) United States
(12) Patent Application Publication
(10) Pub. No.: US 2025/0307836 A1
(43) Pub. Date: Oct. 2, 2025

(54) METHOD AND SYSTEM FOR PROVIDING SUPPORT SERVICES WITH DYNAMIC VOICE PAIRING WITH USERS

(71) Applicant: HUMACH LLC, Frisco, TX (US)

(72) Inventors: Bruce A. Sharpe, Aurora, CO (US); Timothy Houlne, Frisco, TX (US)

(21) Appl. No.: 19/086,668

(22) Filed: Mar. 21, 2025

Related U.S. Application Data
(60) Provisional application No. 63/572,734, filed on Apr. 1, 2024.

(51) Int. Cl. G06Q 30/015; G10L 13/033; G10L 15/00; G10L 15/18; G10L 15/183; G10L 15/22; G10L 15/30; G10L 25/90; H04M 3/51

(52) U.S. Cl. CPC G06Q 30/015; G10L 13/0335; G10L 15/005; G10L 15/1815; G10L 15/183; G10L 15/22; G10L 15/30; G10L 25/90; H04M 3/5183

(57) ABSTRACT

A process for providing support services includes receiving an audio stream from a user device of a user and performing or invoking a voice paring service to perform an audio analysis on the audio stream. The process further includes determining one or more user dimensions about the user based on the audio analysis of the audio stream. The user dimensions include at least certain voice characteristics of the user. A CVI is determined based on the user dimensions using a predetermined algorithm. In one embodiment, a CVI score is calculated to represent the CVI based on the dimension scores of the user dimensions using the predetermined algorithm. Each user dimension may be assigned with a weight factor or coefficient in the formula to represent the fluence of that particular user dimension. The CVI and the user dimensions are then stored in a user profile of the user.

### TECHNICAL FIELD

Embodiments of the disclosure relate generally to contact center technologies. More particularly, embodiments of the disclosure relate to providing supporting services using dynamic voice paring with users.

### BACKGROUND

A contact center is a centralized department within an organization that manages customer interactions across a variety of communication channels, such as phone calls, emails, live chat, social media, and more. It serves as the primary point of contact between a business and its customers, effectively handling inquiries, support requests, complaints, and other customer service functions. The key role of a contact center is to facilitate smooth and efficient communication to ensure customer satisfaction and retention.

The functions of a contact center are diverse and critical to maintaining customer relationships. One of the primary functions is customer support, where representatives assist customers with questions or issues related to products or services. This includes technical support, which involves providing technical assistance and troubleshooting for products, often requiring specialized knowledge. Additionally, contact centers handle sales-related inquiries, process orders, and sometimes make outbound sales calls to engage potential customers.

Many contact centers are now adopting a multichannel communication approach, engaging with customers through various platforms like phone, email, chat, and social media. This approach ensures that customers can reach out through their preferred method of communication. Contact centers can be either in-house, where the organization manages its own operations, or outsourced, where a third-party provider handles customer interactions.

In the competitive landscape of delivering the best customer experience (CX) on voice channels, companies often face limitations by using a single voice for all customer interactions. This approach restricts their ability to personalize interactions, thereby impacting the overall customer experience between the customer and the company.

### BRIEF DESCRIPTION OF THE DRAWINGS

Embodiments of the invention are illustrated by way of example and not limitation in the figures of the accompanying drawings in which like references indicate similar elements.

FIG. 1 is a block diagram illustrating an example of system configuration for providing support services according to an embodiment.

FIG. 2 is a block diagram illustrating an example of a digital agent according to one embodiment.

FIG. 3 is a flow diagram illustrating a processing flow according to one embodiment.

FIG. 4 is a block diagram illustrating examples of user dimensions according to some embodiments.

FIG. 5 is a flow diagram illustrating a process of determining TTS voice and CLMs based on audio analysis of audio streams according to one embodiment.

FIG. 6 is a flow diagram illustrating a process of determining customer voice index according to one embodiment.

FIG. 7 is a block diagram illustrating an example of a user profile according to one embodiment.

FIG. 8 is a block diagram illustrating an example of a customer voice index mapping table according to one embodiment.

FIG. 9 is a flow diagram illustrating a process of voice paring services according to one embodiment.

FIG. 10 is a flow diagram illustrating a process of voice paring services according to one embodiment.

FIG. 11 is a flow diagram illustrating an example of a processing flow according to another embodiment.

FIG. 12 is a flow diagram illustrating a process of voice paring services according to one embodiment.

FIG. 13 is a flow diagram illustrating a process of voice paring services according to one embodiment.

FIG. 14 is a flow diagram illustrating a process of voice paring services according to one embodiment.

FIG. 15 is a block diagram illustrating a data processing system according to one embodiment.

### DETAILED DESCRIPTION

Various embodiments and aspects of the invention will be described with reference to details discussed below, and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present inventions.

Reference in the specification to “one embodiment” or “an embodiment” means that a particular feature, structure, or characteristic described in conjunction with the embodiment can be included in at least one embodiment of the invention. The appearances of the phrase “in one embodiment” in various places in the specification do not necessarily all refer to the same embodiment.

According to some embodiments, customer voice pairing (CVP) is provided, which is a platform designed to enhance personalization in customer interactions by analyzing a customer's voice and pairing it with a similar, though not identical, digital agent voice. This approach aims to create a more personal experience by enabling users to converse with digital agents that sound like them. The system works by identifying several dimensions of the customer's voice in real-time, such as accent, gender, and age. Using these dimensions, the system matches the customer with a digital agent voice that closely resembles their own, fostering a more emotive bond during interactions. For example, a person with a Southern accent from Texas would be paired with a digital agent that reflects similar vocal characteristics. This matching process makes the interaction feel more relatable and engaging.

In addition to voice matching, the platform employs a customer language model (CLM) to further enhance the interaction. This model allows the digital agent to adapt its dialogue based on the identified dimensions of the customer's voice. As a result, the digital agent can use the same abbreviations, slang, and other spoken elements that the customer naturally uses, leading to more authentic and meaningful conversations. By leveraging voice analysis and artificial intelligence (AI), CVP offers a uniquely tailored customer experience that resonates on a personal level.

According to one aspect of the disclosure, a method for providing support services includes several operations. First, a digital agent hosted at a server associated with a contact center receives a first audio stream over a network from a user's device. This audio stream, spoken by the user, contains inquiries about a product provided by a product provider, a client of the contact center. The contact center is designed to offer support services for various products from multiple clients through different communication channels. These clients can be product manufacturers, distributors, retailers, or service providers. The method involves performing an audio analysis on the first audio stream to assess multiple dimensions associated with the user, including voice characteristics like vocal pitch and speech patterns.

A content analysis follows, using the content from the first audio stream. This involves invoking a CLM that has been specifically trained and customized via machine learning for the product in question. The CLM generates a response to the product inquiry. Then, a second audio stream is created based on the response and user dimensions, ensuring that some voice characteristics of this stream are similar to the voice characteristics of the user. Finally, this second audio stream is transmitted back to the user's device over the network.

The method further includes an additional operation of storing the user's dimensions in a user profile on a storage device. These dimensions can be retrieved from the profile to generate responses to future inquiries from the user. The method further includes determining user dimensions by identifying the user's gender and age based on voice characteristics. This ensures that the second audio stream features a voice matching the user's gender and similar age.

Additionally, the method involves determining the user's native language through voice characteristics. The second audio stream is then produced with a voice that matches the user's accent. The further involves understanding the user's intention from the first audio stream. Before content analysis, the method checks if the user's intention matches any predetermined intentions. If a match is found, the corresponding processing flow is initiated to generate the product inquiry response. This processing flow is one among many associated with different intentions, and it can be triggered without invoking the CLM. The CLM is used to produce a response when the user's intention does not match any predetermined intentions.

Performing an audio analysis also involves determining a dimension score for each user dimension. These scores represent the state of each dimension. A customer voice index (CVI) is then calculated based on these scores using a predetermined algorithm, and the CVI is stored in the user's profile. Each dimension score is weighted when calculating the CVI using the predetermined algorithm. The method further includes selecting a text-to-speech (TTS) module from multiple options based on the CVI. Each TTS module can generate a voice with distinct characteristics, and the selected TTS module converts the response into the second audio stream. The method further includes selecting the CLM from various CLMs related to the product based on the CVI. Finally, the method involves converting the first audio stream into a text stream using a speech-to-text (STT) module.

According to another aspect of the disclosure, a method for providing support services involves several key operations. Initially, a digital agent hosted on a server associated with a contact center receives a first audio stream from a user's device during an interactive session. This stream, spoken by the user, contains an inquiry about a product or service offered by a product or service provider—one of the contact center's clients. The contact center is equipped to deliver support services for numerous products from various clients through multiple communication channels. These clients could include product manufacturers, distributors, retailers, or service providers.

Note that throughout this application, the terms of “product” and “service” are exchangeable terms in the contact center space. A product can be physical goods, such as software and/or hardware. A product can also be a service provided by a provider. Similarly, a provider can be a product provider and/or a service provider. Likewise, a contact center can be a premise-based contact center, a virtual/cloud-based contact center hosted by various cloud servers by a third party as part of a platform as a service (PaaS) or contact center as a service (CCaaS), or a combination thereof.

The digital agent then invokes a voice analysis service to conduct an audio analysis of the first audio stream, determining several user-associated dimensions such as vocal pitch and speech patterns. Subsequently, the digital agent uses a custom language model (CLM), specifically trained and customized via machine learning for the product, to generate a response to the user's inquiry. This response is transmitted back to the user's device over the network. The user's dimensions are also stored in a user profile within a storage device, enabling retrieval for generating future responses to subsequent inquiries.

Additionally, the digital agent selects a live agent from a pool of live agents based on the user's dimensions. Each live agent is capable of speaking with different voice characteristics. The interactive session is then transferred to the selected live agent, allowing them to conduct a live session with the user's device regarding the product inquiry. The selected live agent is chosen for their ability to speak with a voice similar to the user's voice characteristics. During the live session, the live agent and the user can communicate with each other via a variety of communications channels, including but not limited to, a voice channel and/or a text channel.

The method further includes generating a second audio stream based on the response and the user's dimensions. This stream is crafted so that its voice characteristics are similar to those of the user. The second audio stream is then transmitted to the user's device over the network, ensuring a more personalized interaction.

The method also includes an additional operation of converting the first audio stream into a text stream using an STT module. This conversion allows the CLM to be invoked on the text stream as input to generate the response. In the method, transmitting the interactive session to the selected live agent involves sending the text stream to the agent. This enables the live agent to review the context of the interactive session during their live interaction, ensuring they have the necessary background information to assist the user effectively.

According to another aspect of the disclosure, a method for providing support services involves several operations. The process begins with a digital agent, hosted on a server associated with a contact center, receiving a first audio stream from a user's device during an interactive session. This audio stream, spoken by the user, contains an inquiry about a product offered by a product provider, who is a client of the contact center. The contact center is equipped to deliver support services for a variety of products from multiple clients through various communication channels. These clients may include product manufacturers, distributors, retailers, or service providers.

The digital agent then invokes a voice analysis service to conduct an audio analysis of the first audio stream, determining several user-associated dimensions such as vocal pitch and speech patterns. Next, the digital agent uses a custom language model (CLM), specifically trained and customized via machine learning for the product, to generate a response to the user's inquiry. This response is then transmitted back to the user's device over the network. The user's dimensions are stored in a user profile within a storage device, enabling retrieval for generating future responses to subsequent inquiries.

The method further includes examining the user's dimensions to determine whether a first predetermined condition has been satisfied, which involves checking if at least one of the dimensions cannot be ascertained. If this condition is met, a notification attribute of the user profile is updated. This attribute is then periodically examined to determine whether a second predetermined condition has been satisfied (e.g., need to hand over to a live agent, detecting a health trait). If the second condition is met, an escalation process is invoked. This includes transmitting an escalate message to a predetermined destination, allowing for the evaluation of potential abnormal dimensions of the user.

According to another aspect of the disclosure, a method for providing support services involves several operations. Initially, a digital agent hosted on a server associated with a contact center receives a first audio stream from a user's device during an interactive session. This audio stream, spoken by the user, is an inquiry about a product offered by a product provider, a client of the contact center. The contact center is designed to provide support services for a wide range of products from multiple clients through various communication channels. These clients could be product manufacturers, distributors, retailers, or service providers.

The digital agent then invokes a voice analysis service to conduct an audio analysis on the first audio stream, identifying various dimensions associated with the user. These dimensions include voice characteristics such as vocal pitch and speech patterns. After this, the digital agent utilizes a CLM, which has been specifically trained and customized through machine learning for the particular product or service, to generate a response to the user's inquiry. This response is then transmitted back to the user's device over the network.

The method also involves storing the user's dimensions in a user profile within a storage device. These dimensions can be retrieved later to generate responses to future inquiries from the user. Additionally, the dimensions are examined to determine if they indicate a health trait concerning the user. This health trait might suggest a likelihood of a health issue associated with the user. If such a health trait is identified, an escalation process is triggered. This process includes sending an escalate message to a predetermined health facility (e.g., a third-party healthcare service provider). The health facility is then responsible for evaluating the health trait, which may involve arranging for medical staff to independently contact the user to discuss the health concern.

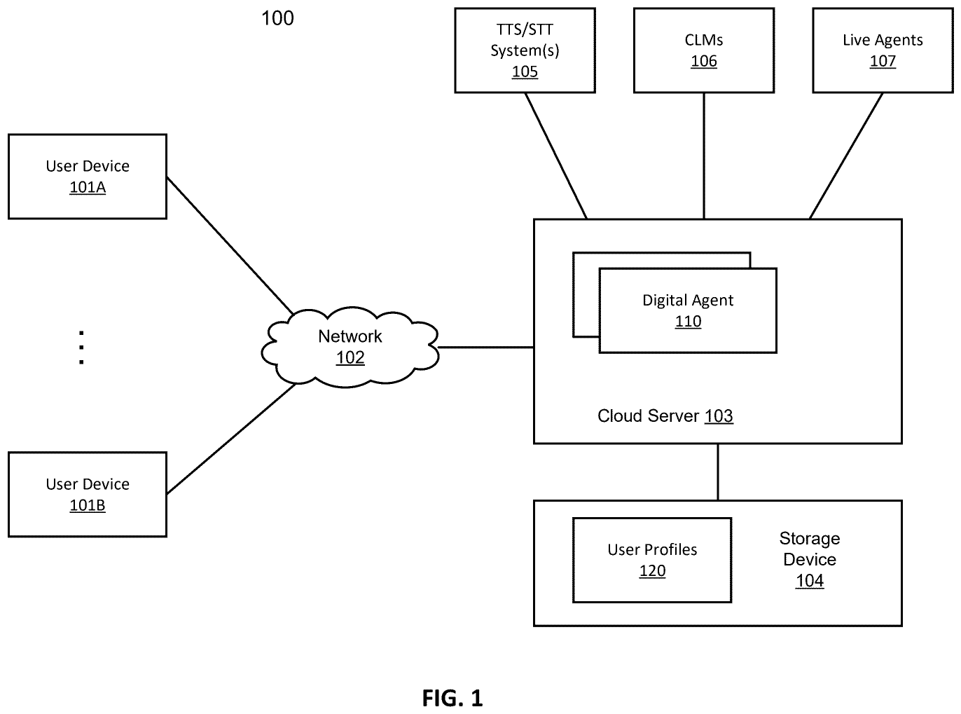

FIG. 1 is a block diagram illustrating a support service system according to one embodiment. Referring to FIG. 1, system 100 includes one or more user devices 101A and 101B (collectively referred to as user devices 101) of users, customer, or individual communicatively coupled to server 103 over network 102. Note that the terms of “user,” “customer,” and “individual” are interchangeable terms throughout this application. Network 102 may be a packet switched network (e.g., local area network or LAN, metropolitan area network or MAN, a wide area network or WAN or Internet), a circuit switched network (e.g., public switched telephone network or PSTN), a voice over IP (VOIP)/session initiative protocol (SIP) communications, or a combination of thereof, wired or wireless. Other network types such as wired or wireless networks for Internet telephony, cellular networks, unlicensed mobile access (UMA) networks, and the like may also be implemented. User devices 101 may be any kind of mobile devices including, but is not limited to, a laptop, mobile phone, tablet, media player, personal digital assistant or PDA, etc. Other devices such as desktops or traditional analog phones may also be utilized by users to contact server 103.

Server 103 may be a part of or associated with a contact center (also referred to as a customer service center or call center) and may be implemented in a centralized facility or server. Alternatively, server 103 may be implemented in multiple facilities or servers in a distributed manner (e.g., cloud-based service platforms such as a CCaaS platform). For example, server 103 may be hosted by a third party on the cloud on behalf of the contact center. Although there is only one server is shown, server 103 may be one of many servers or clusters of servers in various geographic locations or domains in a districted fashion.

Server 103 provides support services to a variety of products or services from a variety of clients or vendors. A client may be a manufacturer, a distributor, a retailer, a service provider or broker, a purchasing facility (e.g., Amazon™), or a combination thereof. In one embodiment, server 103 includes service APIs to communicate with other systems such as systems 105-107, using a variety of network connections or communication protocols. Server 103 may be implemented as a Web server or a frontend server, while other systems 105-107 may be implemented as backend servers.

Server 103 can handle service requests from customers of multiple clients. For example, the contact center may handle customer service requests for a number of retail sales companies, sales calls for catalog sales companies, and patient follow-up contacts for health care providers. In such a structure, the contact center may receive service requests directly from the customers or through client support management systems.

According to one embodiment, one or more digital agents 110 are hosted by server 103 to interact with users of user devices 101 over network 102. Digital agents 110 may invoke services from other systems 105-107 during an interactive session with the users, such as, for example, TTS or SST service, CLM services, and live services from live agents.

A digital agent in a contact center refers to an automated software system designed to handle customer interactions across various communication channels without human intervention. These digital agents may be powered by AI technologies, such as natural language processing (NLP) and machine learning, enabling them to understand and respond to customer inquiries in a conversational manner.

Digital agents provide several key functions in a contact center. One of their primary roles is to deliver automated responses to frequently asked questions or transactions (e.g., booking a plane ticket, looking for status, etc.), which helps reduce wait times for customers. They also offer 24/7 availability, allowing them to provide support at any time of day, unlike human agents who may be limited to specific working hours.

Additionally, digital agents support multichannel interactions, engaging with customers through various platforms like chat, email, voice, and social media. This ensures a seamless experience across different communication channels. By leveraging data from customer interactions and profiles, digital agents can offer personalized recommendations and responses or transactions, enhancing the customer experience.

Moreover, digital agents are valuable for data collection and analysis. They can gather insights from customer interactions, helping businesses understand customer behavior and preferences. These agents are also highly scalable, capable of handling a large volume of interactions simultaneously, which is particularly beneficial for busy contact centers. Overall, digital agents enhance the efficiency and effectiveness of contact centers by automating routine tasks and repeatable complex tasks, allowing human agents to focus on white glove (personalized) support and nuanced customer service issues.

Referring to FIG. 1, when a voice session is initiated between a user and server 103, an audio stream (also referred to as a voice stream) is captured, for example, as an incoming audio stream, and sent to digital agent 110. In response to the incoming audio stream, an audio analysis is performed to determine content of the audio stream and user dimensions of the user that present the user.

A “user dimension” refers to specific characteristics or attributes of a user (typically a customer) that can be analyzed and utilized to enhance interactions and personalize service. These dimensions help the contact center understand the user better and tailor responses or services accordingly.

Common user dimensions include voice characteristics, such as vocal pitch, tone, and speech patterns. Analyzing these can help create a more personalized interaction, especially when using voice-based digital agents. Additionally, demographic information like age, gender, and location can be considered dimensions that assist in tailoring communication styles and service offerings.

Behavioral patterns are another important dimension, referring to how a user interacts with the contact center. This includes their preferred communication channel (chat, phone, email), the frequency of contact, and common inquiries or issues. User preferences and history, including past interactions, purchase history, and preferences, can be used to anticipate needs and provide more relevant support or recommendations.

Sentiment analysis is also a valuable dimension, as it involves analyzing the sentiment or emotional tone of a user's communication. This can help in adjusting the response strategy to better meet the user's current emotional state. Finally, understanding the user's primary language and regional accent can assist in providing more accurate and relatable communication. By understanding and utilizing these dimensions, contact centers can improve customer satisfaction by offering more personalized, efficient, and effective service.

Based on the user dimensions, a customer voice index (CVI) is determined using a predetermined algorithm. The CVI associated with the user is then saved in a user profile 120 and stored in storage device 104. User profile 120 may be implemented as a part of user database. Storage device 104 may be implemented as a storage server, maintained locally or remotely over a network.

In one embodiment, based on at least some of the user dimensions (e.g., user intention), a proper CLM may be identified and selected to generate a response to the user inquiry. For example, based on the CVI, a corresponding CLM is selected from an array of CLMs designed for a variety of situations or schemes. A CLM is specifically configured to handle the interactive sessions or conversation flows of a client the contact center represents.

In one embodiment, once the response is generated using the selected CLM, digital agent 110 invokes a TTS system 105 to convert the response into an outgoing audio stream. The TTS system or module may be identified and selected from an array of TTS systems or modules, each corresponding to a specific voice with specific voice characteristics. In an embodiment, the TTS system may be selected based on the CVI associated with the user. As a result, the outgoing audio stream may have a voice similar to the voice of the user.

According to another embodiment, under certain circumstances, when a live agent is needed, a live agent may be selected from a pool of live agents of the contact center based on the user dimensions of the user. For example, a live agent may be selected based on the CVI of the user. As a result, a live agent having the similar voice or speaking the same language of the user may be selected to handle the live session with the user, and the user would feel more comfortable and have better customer experience.

According to a further embodiment, based on the audio analysis, certain conditions (e.g., triggering conditions) may be determined. If a particular condition is satisfied, an alert or a notification message may be transmitted to a predetermined destination. For example, based on the audio analysis, if it is determined that the user is not satisfied with the response or the user repeats the same or similar questions, it may be determined that it is time to escalated, for example, invoking a live agent. Alternatively, if certain user dimensions cannot be determined, a notification or escalation may be triggered.

In another embodiment, based on the audio analysis, if a health trait concerning the user is identified, the situation may be escalated and a health facility may be contacted to allow a health professional to reach out to the user to discuss a potential health issue of the user. The above configurations may be specifically defined as part of the user profile 120 of the user.

A “health trait” refers to characteristics or indicators related to the physical or mental well-being of a user, which can be inferred or directly observed through their interactions with the contact center. These traits are not common but can be especially relevant in sectors like healthcare, wellness, or insurance services, where understanding a customer's health status can be crucial for providing appropriate support or recommendations.

Health traits might be applied or identified in a contact center setting through several methods. Advanced voice analysis technologies can sometimes detect stress, fatigue, or emotional distress in a user's voice, suggesting potential health issues or the need for immediate support. Additionally, the content of interactions can reveal health-related concerns. For example, a customer might discuss symptoms, ask about medication, or express anxiety about a health condition.

Interaction patterns can also provide insights, as frequent contact with a health-related customer service line might indicate ongoing health issues. Patterns such as increased frequency or urgency of calls could signal changes in a user's health status. In contact centers connected to healthcare services, user profiles might include health traits derived from medical records or previous healthcare interactions, which can be used to tailor the support provided.

Understanding the emotional state of a user through sentiment analysis could highlight mental health concerns, prompting the contact center to offer additional support or escalate the issue to a healthcare professional if necessary. By identifying and understanding health traits, contact centers can provide more empathetic, personalized, and effective support, ensuring that users receive the care and attention they need, particularly in sensitive or urgent situations.

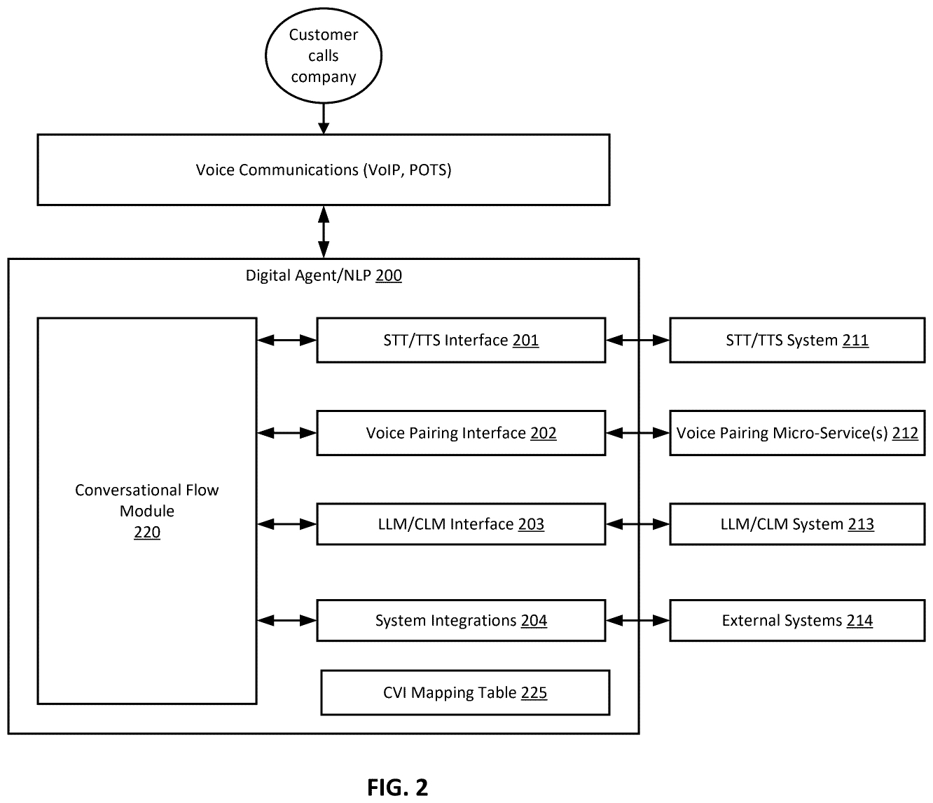

FIG. 2 is a block diagram illustrating an example of a digital agent according to one embodiment. Digital agent 200 may represent any of digital agents 110 of FIG. 1. Referring to FIG. 2, when a customer or user initiates a voice session with the contact center, digital agent 200 launches conversational flow 220 to interact with the user to collect certain voice samples of the user for the purpose of voice paring. The conversational flow 220 was configured to have a predetermined set of questions to be asked the user. Based on the user's responses, the voice samples are captured. Conversational flow 220 then invokes voice paring service 212 via the corresponding interface 202. Note that conversational flow 220 can invoke a variety of services such as 211-214 via corresponding interfaces 201-204 (e.g., application programming interfaces or APIs).

Based on the captured voice samples, voice paring service 212 is configured to perform an audio analysis to determine user dimensions of the user. Based on the analysis, a user dimension score is calculated for each user dimension. A CVI score is then calculated based on the user dimension scores using a predetermined algorithm. In calculating a CVI score, each user dimension is associated with a weight factor. The CVI score is then stored in a user profile of the user. The CVI score may be used as an index to determine an STT or TTS system and a CLM model to be used to generate responses to the user. In an embodiment, digital agent 200 maintains a CVI mapping table 225 that maps various CVI scores to other components such as TTS systems or CLMs.

Before engaging with a digital agent or proceeding to a live agent interaction, customers are asked to answer a few questions. This process allows for the collection of voice samples for quick analysis. Customers are identified through an authorization process established by the company to verify their identity. If the customer is new, they will be guided through a sign-up process to create a unique customer profile. However, if they choose not to sign up, an account profile is still created using standard information such as their phone number or data collected from a survey.

Once the customer's identity is verified through authorization, a few seconds of their voice are required at the start of an interaction to analyze and determine their dimensions. This unique service can be integrated into any digital or live agent platform. The analysis helps in pairing both digital and live agents with the customer more effectively.

This service supports two modes of analysis. The first mode involves analyzing the interaction's start, using specific questions to gain samples for analysis. These questions should be seamlessly integrated into the interaction's greeting and introduction, aligning naturally with the customer's support needs. The second mode involves analyzing the entire interaction, using audio throughout the session.

Once the analysis is complete and dimensions are identified, the customer will be presented with profile settings specific to their CVI score during their next interaction with the company. In mode two, if specific dimensions, such as “health traits,” are identified, they may trigger a notification or escalation to a third-party service. Other identified dimensions, like voice and CLM, will take effect in subsequent interactions with the customer.

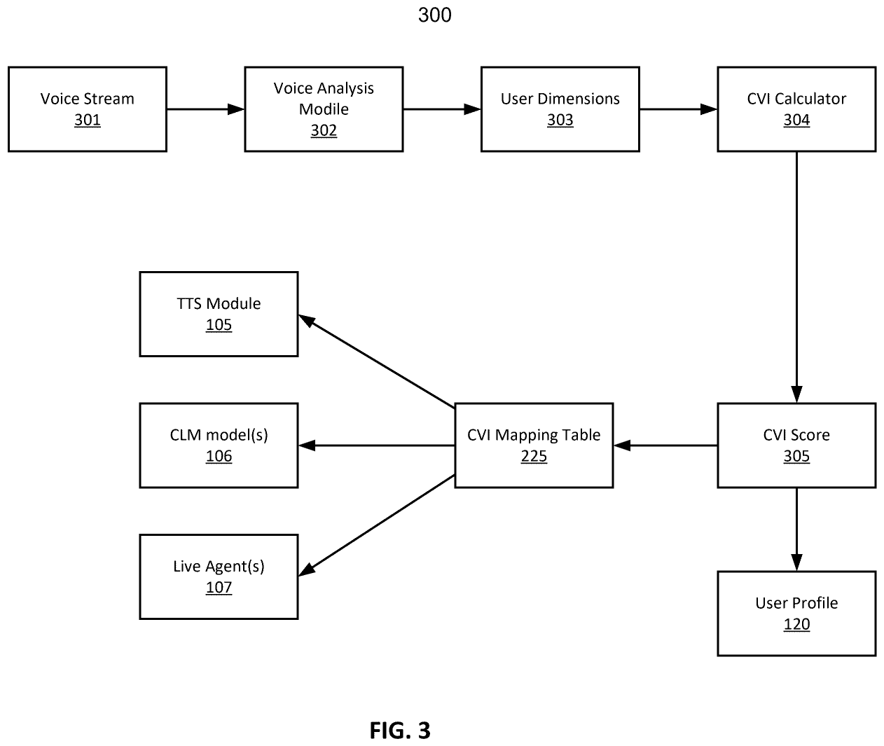

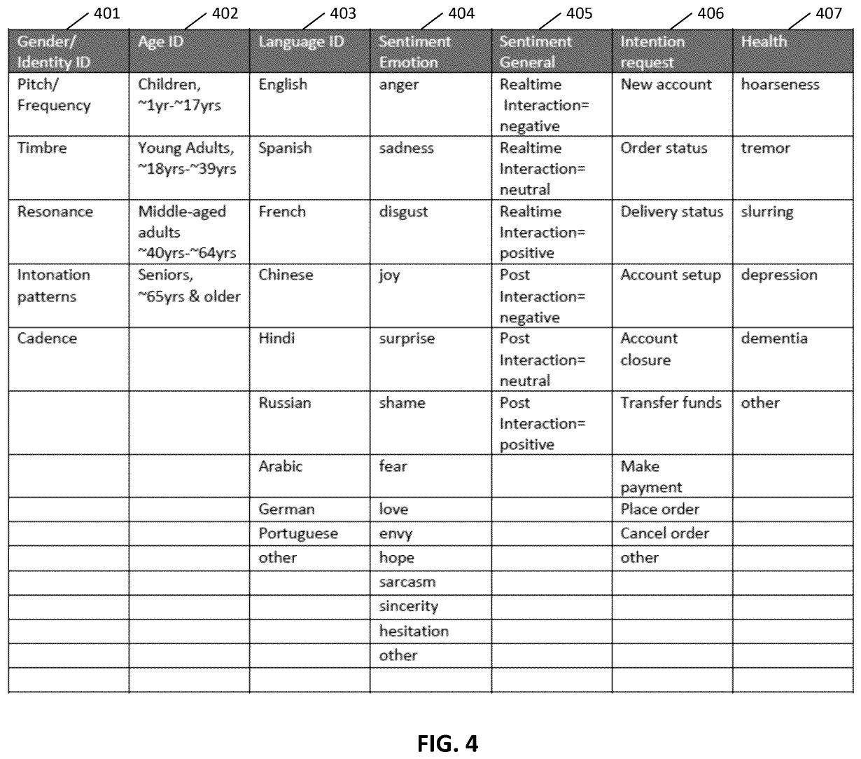

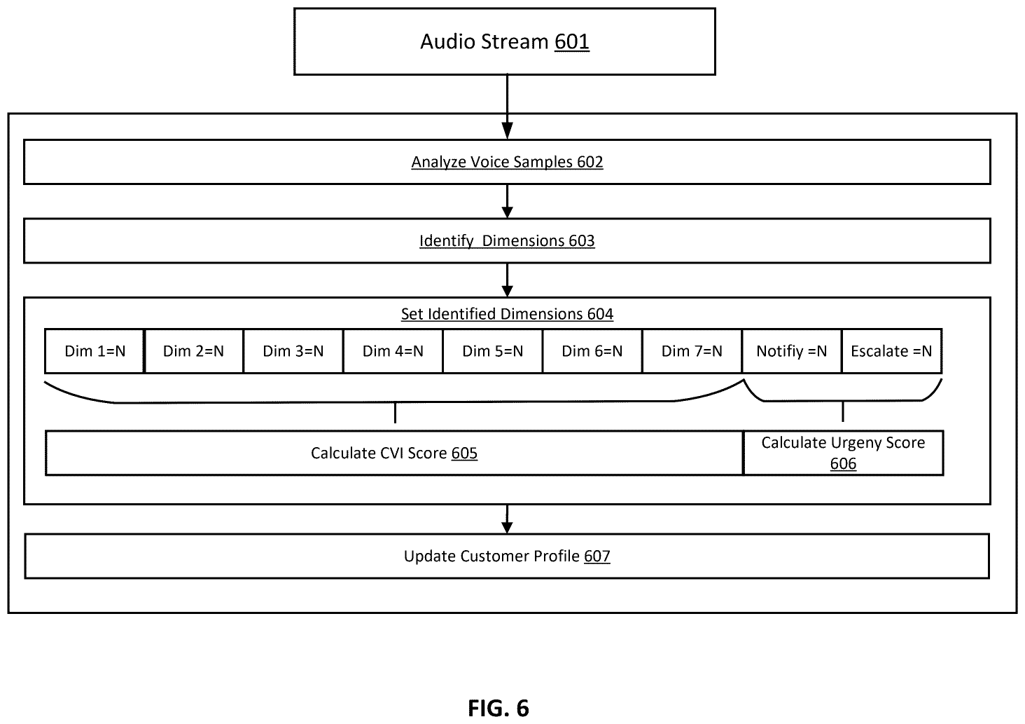

FIG. 3 is a flow diagram illustrating a processing flow according to one embodiment. Referring to FIG. 3, processing flow 300 starts with a voice stream 301 captured from a user when the user initiates a voice session with a contact center. The voice stream (or its voice samples) is then fed into a voice analysis module or service 302 to determine user dimensions 303. At least some of the user dimensions are shown in FIG. 4 according to one embodiment. Each user dimension is assigned with a dimension score based on the audio analysis. A process of determining user dimensions is shown in FIG. 6.

Referring to FIG. 6, when an audio stream 601 is received, an audio analysis is performed on the audio stream at block 602. User dimensions are identified at block 603 based on the audio analysis. In this example, user dimensions 604 include dimensions 1 to 7. Each dimension is assigned with a dimension score based on the audio analysis. The dimension scores are then utilized to calculate a CVI score 605 using a predetermined algorithm. In addition, a notification score and an escalation score may also be determined. An urgency score 606 is calculated based on the notification score and escalation score. Thereafter, the CVI score 605, urgency score 606, as well as the user dimensions are stored in the user profile of the user.

In one embodiment, a notification score and an escalation score may be determined based on the user dimensions. Under certain circumstances, based on the user dimensions, there is a need to notify a support staff of the contact center. For example, if some of the user dimensions, such as intent 404, cannot be determined based on the audio analysis, the notification score may be calculated based on how severe or importance of the corresponding dimension (e.g., relative to its weight factor). If the notification satisfies a predetermined condition (e.g., higher than a threshold), the system may collect the information pertinent to the user (e.g., interactive history or transcript produced by a STT system), and send a notification to a support staff to allow the support staff to determine the intent in real time. Alternatively, if a CLM corresponding to the CVI 605 cannot be determined, a notification may be triggered such that a support staff can determine the CLM in real time.

In addition, an escalation score may be calculated based on some of the dimensions. For example, based on sentiment score 405 and health score 407, the system may determined there is a need to escalate the session to a health center. As a result, information pertinent to the user is collected and transferred to the predetermined facility. The escalation process may also be logged in the user profile as a part of interactive history. Based on the notification score and/or escalation score, urgency score 606 is calculated using a predetermined algorithm. Urgency score 606 may be utilized to determine the priority or urgency of the notification or escalation process for scheduling purpose.

In some embodiments, several key dimensions can be identified from a customer's voice sample during an interaction. Referring to FIG. 4, one such dimension is gender identity 401, where the system analyzes vocal pitch, tone, and speech patterns to estimate whether the speaker's gender is male or female, despite some overlap and variation. Another dimension is age 402, where certain vocal characteristics help estimate the speaker's age range. Although an exact age cannot be pinpointed, the system can classify the speaker within a specific age window by examining vocal pitch, tone, and speech patterns, with reference to the table mentioned earlier.

Language and accent 403 are also crucial dimensions. The system can identify a speaker's regional background or native language, such as Spanish, French, or Japanese, through STT transcription when they speak in their native language. If a native Spanish speaker communicates in English, the system can prompt them to confirm their preferred language, initiating a language identification process and updating their profile accordingly.

The analysis includes determining sentiment 404 and emotion 405 as part of user dimensions, which are assessed in real-time and post-interaction. General sentiment is categorized as positive, neutral, or negative, while enhanced emotion detection can identify feelings such as happiness, sadness, anger, and frustration.

Understanding the intention 406 behind a customer's words is vital for grasping their needs. The system identifies key phrases like “new account,” “order status,” and “delivery status” to interpret the customer's purpose. For instance, saying “I would like to open a new account for my son” indicates an intent for a “new account,” while “I want to check the status of my order” corresponds to an “order status” intent.

The purchasing history dimension involves tracking a customer's spending over various periods (day, week, month, year), helping to assess their financial value and likelihood of future purchases. Similarly, repeat contact history examines how often customers contact the business, which could indicate unresolved issues and impact metrics on customer support effectiveness.

Finally, the dimension of health 407 is considered, as certain health conditions can affect voice characteristics. Identifying these can prompt the system to suggest medical attention. By understanding these dimensions, contact centers can tailor interactions more effectively and provide personalized service.

When determining the user dimensions, each dimension is assigned with a dimension score or value representing the state or status of the dimension. For example for language dimension 403, each language may be assigned with a unique dimension score representing the type of language. Similarly, for intention dimension 406, different dimension scores may be utilized to represent different intentions. These dimensions scores may be utilized to calculated a CVI using a predetermined algorithm. Each type of dimension may be associated with a weight factor or coefficient representing the influence of the corresponding dimension in determining the CVI.

Each dimension is critical in creating the proper pairing of a customer to a digital agent voice and CLM. Results of these dimensions are used to define a CVI. The CVI is used as an ID mapped to a specific TTS voice of the digital agent. It will also be mapped to a specific dialog model to help the digital agent to speak in the same manner as the customer.

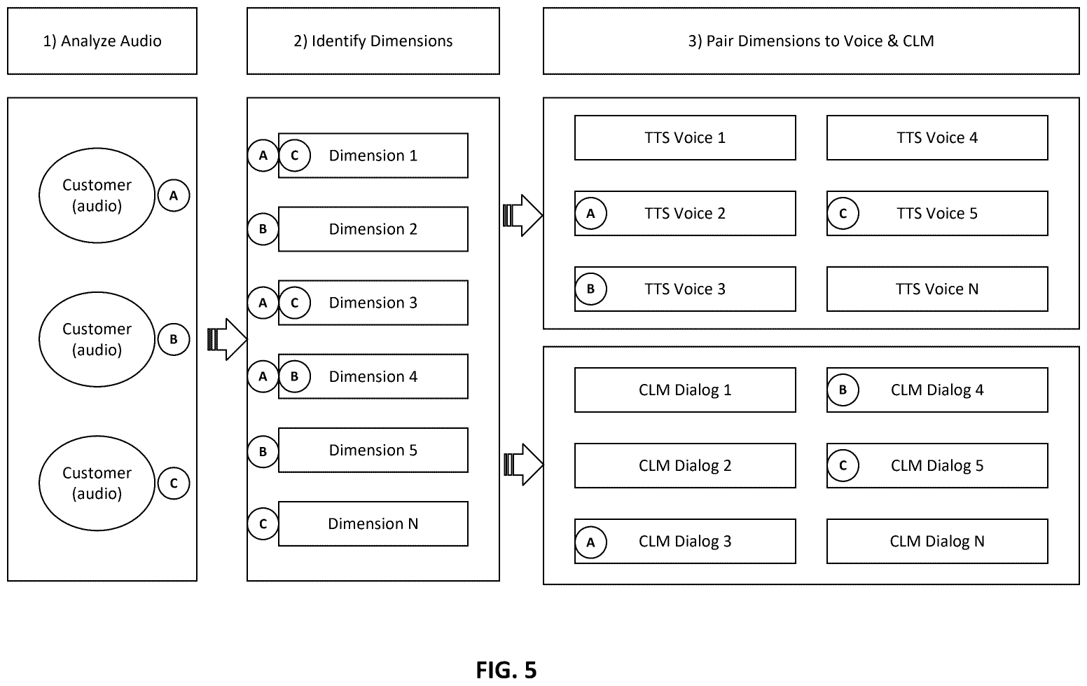

Referring back to FIG. 3, user dimensions 303 are then used by CVI calculator 304 to determine a CVI score 305 using a predetermined algorithm, an example process of which is shown in FIG. 5. Referring to FIG. 5, in this example, audio streams A, B, and C associated with customers A, B, and C are captured when the customers contact the contact center. Audio streams A, B, and C are analyzed to determine user dimensions. In this example, dimensions 1 and 3-4 are identified for customer A; dimensions 2 and 4-5 are identified for customer B; and dimensions 1, 3, and N are identified for customer C. Based on the user dimensions associated with the customers, TTS voice 2 and CLM 2 are selected for customer A and stored in the user profile of customer A. Similarly, TTS voice 3 and CLM 4 are selected for customer B, and TTS voice 5 and CLM5 are selected for customer C.

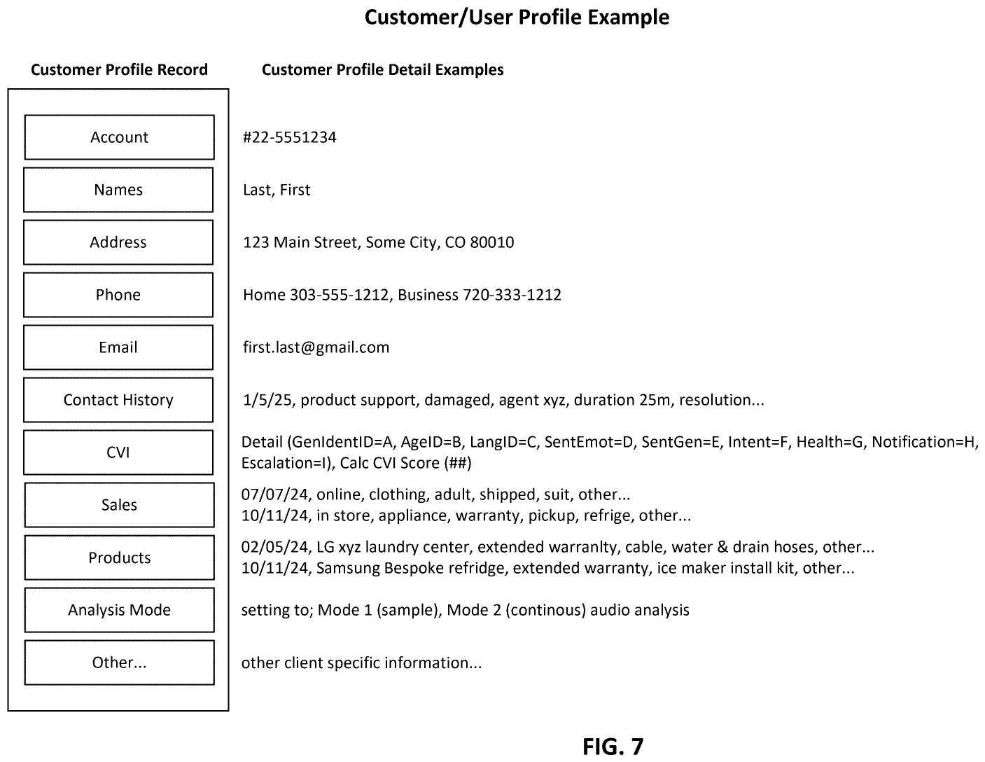

CVI calculator 304 and voice analysis module 302 may be provided as part of voice pairing service 212. Alternatively, they may be maintained as a part of a digital agent. Both user dimensions 303 and CVI score 305 may be stored in user profile 120 of the user. An example of a user profile is shown in FIG. 7.

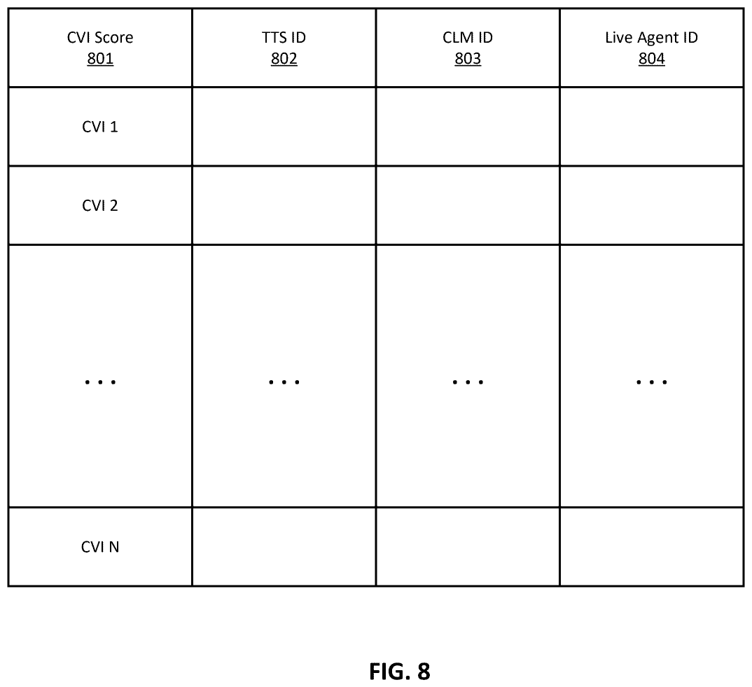

In addition, CVI score 305 may be used as an index to CVI mapping table 225 to identify other corresponding components such as TTS system 105, CLM model 106, and live agents 107. An example of a CVI mapping table is shown in FIG. 8, which may be stored in a storage device accessible by the digital agent. Referring to FIG. 8, given a CVI score 801, a TTS 802 with a specific voice can be identified. In addition, an associated CLM 803 can also be identified. In case a live agent is needed, a live agent 804 with similar voice can also be identified.

Referring to FIGS. 2 and 3, the dynamic voice paring platform is composed of several integral system components that work in unison to facilitate seamless interactions between digital agents and customers. The first component is a digital agent, which enables businesses to construct conversational flows that interact with live customers using technologies such as natural language processing (NLP), STT, and TTS. These technologies convert audio conversations into text and back into audio. To enhance the naturalness of these interactions, a large language model (LLM) can be integrated, allowing for dynamic conversations. This platform is designed to interface with various systems through APIs, supporting essential transactions like checking account balances, transferring funds, and tracking orders.

The SST is another crucial component, specializing in converting spoken words into text. This conversion enables the digital voice agent to understand and process conversations using NLP. Conversely, the TTS converts text into speech that mimics the human voice, allowing the digital agent to audibly communicate new messages to customers.

The platform also incorporates a foundation models, which are trained on extensive text data. These AI programs, built on machine learning techniques and utilizing transformer neural networks, can recognize and generate text. To further refine these models, LLM tuning involves adjusting the model's parameters to suit specific tasks through specialized training data or content unique to a company, resulted in CLMs. This customization can include industry-specific terms and slang to improve communication.

Additionally, the platform includes voice analysis or voice paring, custom services designed to analyze audio streams or recordings to determine specific dimensions of customer conversations. The mode of analysis is determined by a “mode” setting in the customer profile, and the results are stored for use in tailoring interactions, such as selecting a TTS voice or adapting CLM dialogue. These services incorporate custom algorithms for processing dimensions.

Together, these components form a comprehensive solution capable of handling both incoming and outgoing customer interactions. The system requires setup, build, and configuration for each customer to tailor the platform to their specific needs and use cases.

Customer personalization hinges on the capabilities of voice analysis and pairing microservices. These microservices conduct real-time audio analysis to evaluate and identify specific dimensions of a customer's voice. This analysis serves several purposes: identifying a specific TTS voice that matches the customer's preferences, tailoring a CLM dialog for a more personalized and relevant conversation, and optionally using a company-defined cloned TTS voice for branding purposes to maintain brand consistency.

Once these dimensions are identified from a customer audio sample, whether using mode 1 or mode 2, they are utilized to create and update a CVI. The CVI is crucial for dynamically transforming the digital agent into a persona that closely resembles the customer, aligning with the support expectations defined by the company in the digital agent and CLM.

After the CVI is established, it is added to the customer's profile record for future interactions with the company's digital agents. This profile also includes a setting to determine whether mode 1 or mode 2 should be used, as shown in FIG. 7. This setting is customized for each customer, ensuring a consistent and personalized experience in every interaction.

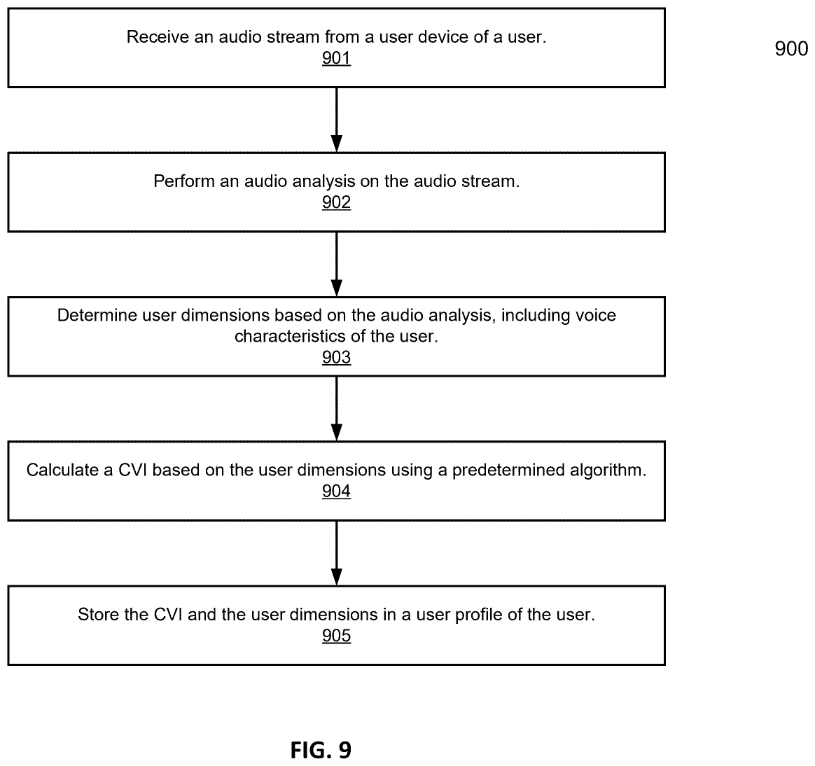

FIG. 9 is a flow diagram illustrating a process of voice paring services according to one embodiment. Process 900 may be performed by processing logic, which may include software, hardware, or a combination thereof. For example, process 900 may be performed by a digital agent, a server, and/or a contact center. Referring to FIG. 9, at block 901, processing logic receives an audio stream from a user device of a user. The audio stream was spoken by the user, for example, via a microphone of a mobile device. In the audio stream, the user may inquire about a product or service of a client of the contact center. At block 902, processing logic performs or invokes a voice paring service to perform an audio analysis on the audio stream. At block 903, processing logic determines one or more user dimensions about the user based on the audio analysis of the audio stream. The user dimensions include at least certain voice characteristics of the user, such as, for example vocal pitch and speech patterns of the user. In one embodiment, for each of the user dimensions, a dimension score is calculated and assigned to the corresponding dimension.

At block 904, a CVI is determined based on the user dimensions using a predetermined algorithm. In one embodiment, a CVI score is calculated to represent the CVI based on the dimension scores of the user dimensions using the predetermined algorithm. Each user dimension may be assigned with a weight factor or coefficient in the formula to represent the fluence of that particular user dimension. At block 905, the CVI (e.g., CVI score) and the user dimensions (e.g., dimension scores) are then stored in a user profile of the user. The CVI and the user dimensions may be utilized subsequently to identify the proper TTS voice, CLMs, and/or live agents to improve user experience. Note that process 900 may be performed when the user is new to the system, i.e., first time login. In this situation, the user's voice is unknown to the system. Process 900 is utilized to determine the user's voice for subsequent better service and customer experience.

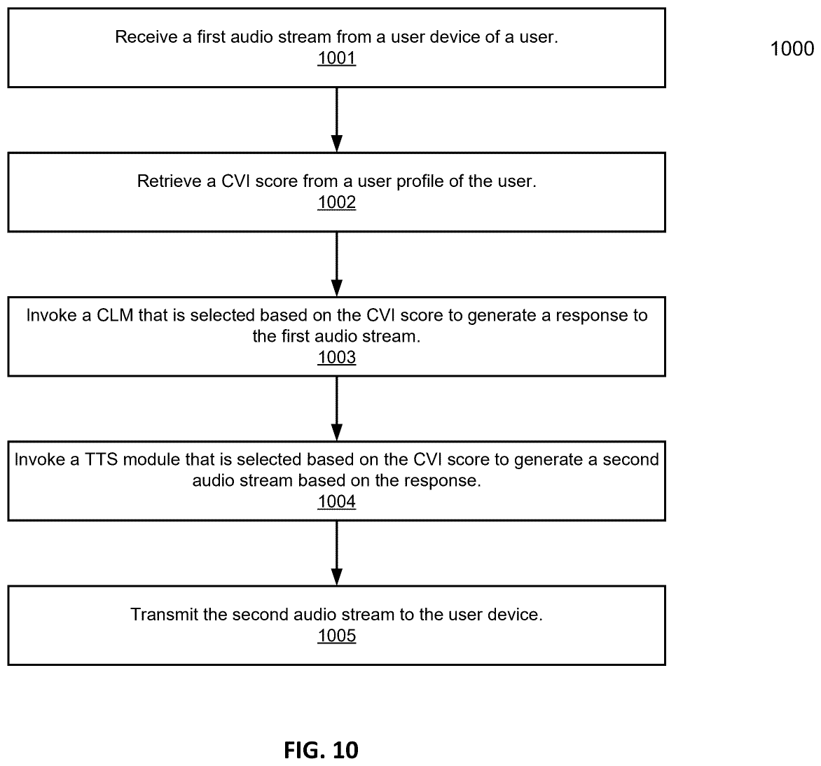

FIG. 10 is a flow diagram illustrating a process of voice paring services according to one embodiment. Process 1000 may be performed by processing logic, which may include software, hardware, or a combination thereof. For example, process 1000 may be performed by a digital agent, a server, and/or a contact center. Process 1000 may be performed after the CVI and user dimensions of the user have been ascertained, for example, via process 900. Referring to FIG. 10, at block 1001, processing logic receives an audio stream from a user device of a user. The audio stream was spoken by the user, for example, via a microphone of a mobile device. In the audio stream, the user may inquire about a product or service of a client of the contact center. At block 1002, processing logic retrieves a CVI from a user profile of the user, where the CVI may be previously determined via voice pairing, for example, via process 900.

At block 1003, processing logic invokes a CLM that is selected based on the CVI retrieved from the user profile, for example, via a CVI mapping table as described above. The processing logic generates a response to the first audio stream using the CLM. In an embodiment, the first audio stream may be converted into a text stream using a STT system prior to applying the CLM. At block 1004, processing logic invokes a TTS system that is selected based on the CVI to generate a second audio stream. The selected TTS system is configured to generate an audio stream having voice characteristics corresponding to the CVI (e.g., similar to the voice characteristics of the user). At block 1005, the second audio stream is transmitted as a response to the user device.

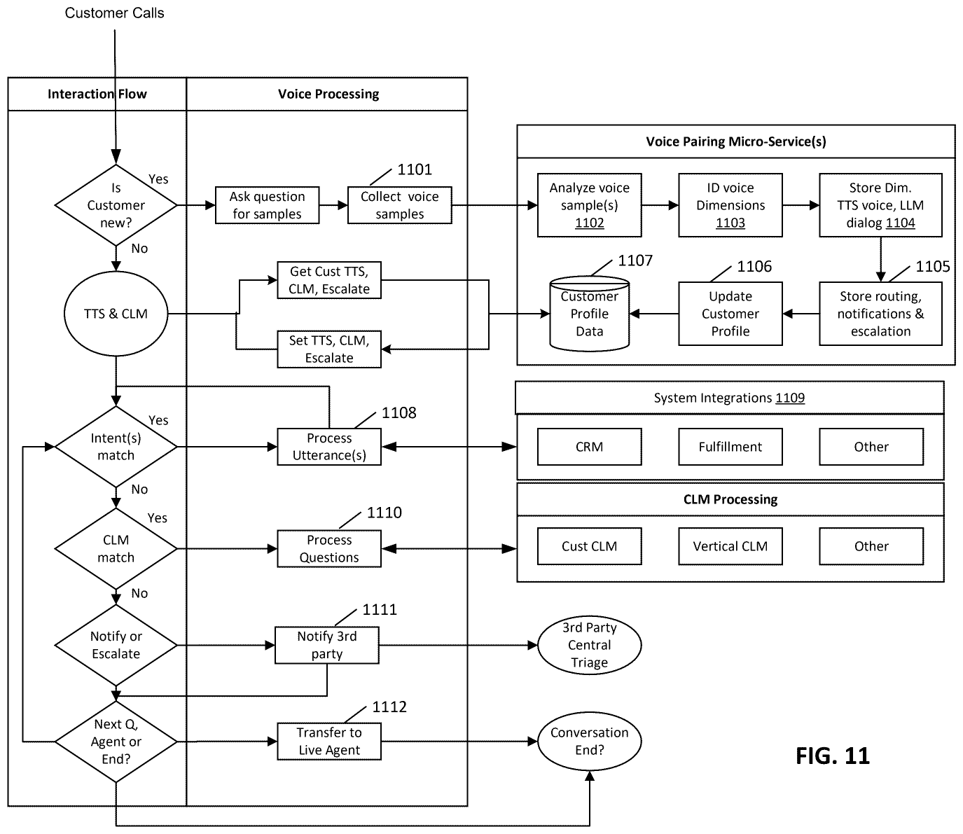

FIG. 11 is a flow diagram illustrating an example of a processing flow according to another embodiment. Referring to FIG. 11, when a customer places a call to a business number, the business receives this inbound call and it is promptly answered by a digital voice agent. The digital voice agent employs STT technology to transcribe the customer's voice, allowing for seamless interaction with the customer. Note that throughout this application, “user” and “customer” are interchangeable terms.

If the customer is new and calling for the first time, the system queries the customer profile to determine which analysis mode to use, either “mode 1” or “mode 2.” If “mode 1” is selected, the digital agent asks a series of questions to collect audio samples at block 1101. These questions are tailored to the specific customer project and might include queries such as “What can I assist you with?” and “Have you contacted us previously about this issue?” For “mode 2,” audio samples are collected continuously throughout the interaction.

As audio samples are gathered, they are immediately sent to the voice pairing microservice for analysis at block 1102; the system does not wait for all samples to be collected before proceeding. In “mode 2,” the system can process audio and utterances in the background, identifying traits such as health indicators. All collected audio samples are analyzed to determine specific dimensions at block 1103, which are then used to map the appropriate responses and actions.

Once the dimensions are identified, they are processed through an algorithm to determine the best pairing of voices, CLM/LLM, routing, notifications, and any necessary escalations for the interaction at blocks 1104-1105. This process results in the creation of a CVI, which defines the platform resources to be used in both current and future interactions with the customer.

The CVI is used to set the TTS voice and LLM. It also informs interaction routing, notifications, and escalations. The CVI results are stored in the customer profile for use in future interactions at block 1106. Whether dealing with a new or existing customer, the system processes the information and then makes a call to the customer profile database 1107 to retrieve the profile record, including the CVI results. This ensures that each interaction is informed by the most up-to-date customer data.

After setting the TTS, CLM, notifications, or escalations, the digital agent evaluates the customer's intent. If an intent is determined, the system uses the CVI results to set the appropriate TTS voice. The system processes the identified intent and streams responses from the digital agent to the customer in the selected TTS voice at block 1108. Once the flow is complete, the digital agent asks the customer if there is anything else they need assistance with. If no intent is identified, the system moves to the next step in processing. Additionally, the intent can trigger connections to various systems 1109 via flow processing to gather any additional information required.

The system then passes the interaction to the CLM/LLM for further processing. Using the CVI, the system calls upon the appropriate CLM/LLM at block 1110. The customer's utterance is sent to the CLM/LLM, which processes the input and sends a response back to the digital agent. This response is then streamed to the customer using the set TTS voice. The CLM/LLM utilized can be custom, vertical, fine-tuned, or another type as needed for processing the customer's needs.

The system checks whether any notifications or escalations are required. If there are, a request is sent to a third-party central triage or support center at block 1111, and system processing continues. Once the interaction stream is complete, the digital agent prompts the customer with a follow-up question or the next question.

Based on the customer CVI, notifications to support staff can be established to identify issues or the lack of support for one or more dimensions identified. Using one-time or ongoing processing during an interaction of CVI dimensions, the system can perform notifications at any time.

These notifications can be based on, but are not limited to, several factors: unavailable or unidentified voice needed to support the customer, unavailable CLM/LLM or content needed to support the customer, and improper identification of one or more of the customer's dimensions. These dimensions might include age identification, language accent, identity, intention, and health trait.

The result of these notifications provides real-time monitoring of the interactions and guides in the system enhancements of necessary system components. These components include system classifiers, TTS voice models, CLM/LLM content or models, intent identification, and system processing logic. Other tuning may be necessary to further enhance the customer's ongoing experience.

Based on the CVI and identified notification urgency score, the proper escalations for customer interactions can be performed. The company can establish the necessary escalation processing for a customer by setting flags on CVI dimensions for a company project. If an escalation is identified, a path established by the company can be executed.

Examples of these escalations include live white glove support for multi, complex, or other intents, live problem solving on purchase or repeat interactions, performing language identification, health trait identification handling, and other specific needs.

The result of these escalations provides real-time monitoring of the interactions and guides in the system enhancements of necessary system components. These components include system classifiers, TTS voice models, CLM/LLM content or models, intent identification, system processing logic, and live agent routing. Additional tuning may be necessary to further enhance the customer's ongoing experience.

The system can also perform an escalation to live agent routing if required and configured for the company. Although most interactions are moving towards digital self-service agents, there remains a need, which is growing, for live voice support for customers. Other live channels, such as chat and SMS, are supported but at a smaller volume level.

Depending on the company's clientele or industry, live agents may be optionally supported or required as a point of escalation out of a digital agent interaction. In these situations, the CVI is utilized to perform routing similar to those outlined in the “escalation” section.

If necessary, based on company policy, the digital agent may transfer the interaction to a live agent at block 1112. If a request for a live agent is made, the system initiates the transfer of the customer interaction to a live agent with the appropriate skill set. The interactive session is transferred to a queue associated with the selected live agent for pending to be processed by the live agent. The live agent is selected based on the CVI stored in the customer's profile, ensuring that the transfer maintains a consistent customer experience. The system uses CVI matching to route the customer to a suitable live agent, thereby preserving the personalized service experience.

A live agent is a human representative who interacts directly with customers to address their inquiries, resolve issues, and provide support. Live agents are trained to handle a wide range of customer service tasks, including answering questions, processing orders, troubleshooting technical problems, and managing complaints. They typically communicate with customers through various channels such as phone calls, live chat, email, and sometimes even social media. Live agents play a crucial role in providing personalized and empathetic service, ensuring customer satisfaction and fostering positive relationships between the company and its clients.

One of the situations that involves an escalation process is detecting a health trait of the user based on the audio analysis. While the system does not diagnose or recommend treatment to the customer, health traits identified can be sent to be acted upon by a trained professional. An example of this process via a notification is as follows:

First, a customer interacts with the system. The system then processes the new or existing customer and identifies one or more health trait indicators. System configuration can be set up to identify the communication type. For example, a notification might be sent if a health trait is identified in one or two interactions, while an escalation may occur if a health trait is identified in many interactions. This can also be based on frequency, such as daily, weekly, monthly, or yearly.

A notification is then sent to a central health center for review, which includes the communication type, one or more identified health traits, and the frequency of identified health traits. A certified health specialist or doctor is made aware of the communication. The specialist or doctor then performs an assessment of the communication to identify a course of action. This may include outreach to the customer's primary care physician on file, reviewing available health records to assist in action, or performing outreach to the customer based on any identified concerns the medical doctor might have regarding the current health status.

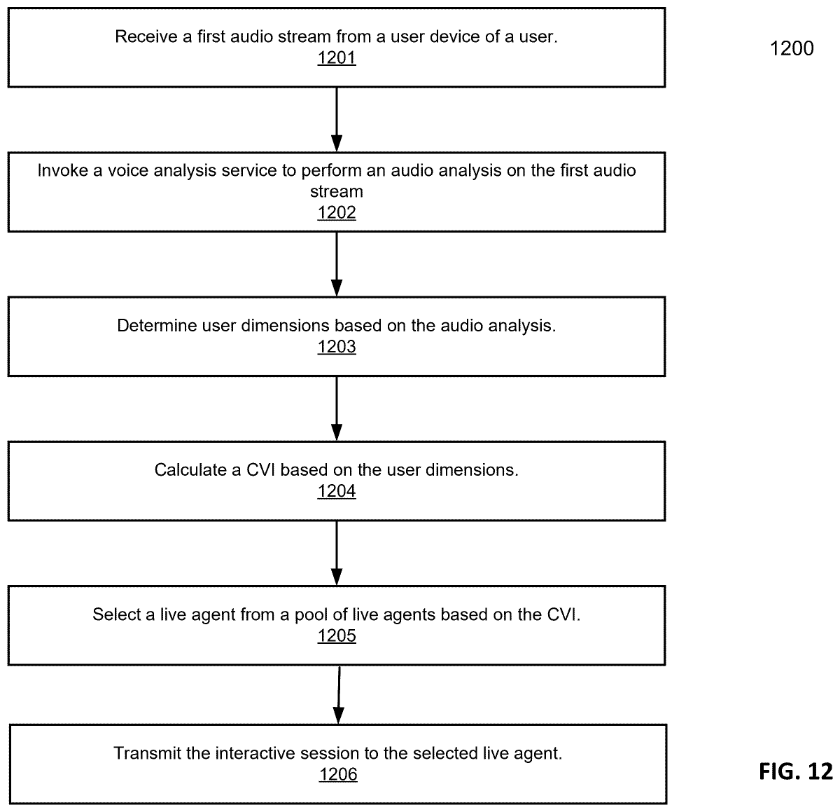

FIG. 12 is a flow diagram illustrating a process of voice paring services according to one embodiment. Process 1200 may be performed by processing logic, which may include software, hardware, or a combination thereof. For example, process 1200 may be performed by a digital agent, a server, and/or a contact center. Referring to FIG. 12, at block 1201, processing logic receives an audio stream from a user device of a user. The audio stream was spoken by the user, for example, via a microphone of a mobile device. In the audio stream, the user may inquire about a product or service of a client of the contact center. At block 1202, processing logic invokes a voice analysis service to perform an audio analysis on the first audio stream. At block 1203, processing logic determine user dimensions of the user based on the audio analysis. At block 1204, processing logic calculates a CVI based on the user dimensions. At block 1205, processing logic selects a live agent from a pool of live agents based on the CVI. For example, the live agent can be selected based on a CVI score via the CVI mapping table as described above. As a result, the selected live agent would have a voice similar to or compatible with the voice characteristics of the user. At block 1206, processing logic transmits the interactive session to the selected live agent to allow the live agent to conduct a live session with the user. During the live session, the live agent and the user can communicate with each other via a variety of communications channels, including but not limited to, a voice channel and/or a text channel.

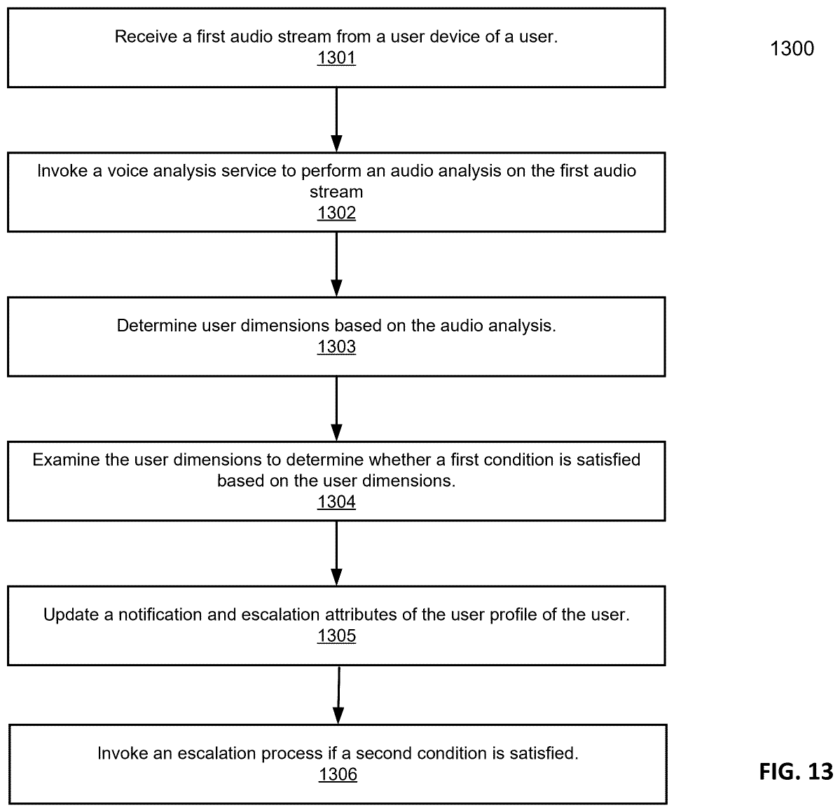

FIG. 13 is a flow diagram illustrating a process of voice paring services according to one embodiment. Process 1300 may be performed by processing logic, which may include software, hardware, or a combination thereof. For example, process 1300 may be performed by a digital agent, a server, and/or a contact center. Referring to FIG. 13, at block 1301, processing logic receives an audio stream from a user device of a user. At block 1302, processing logic invokes a voice analysis service to perform an audio analysis on the first audio stream. At block 1303, processing logic determine user dimensions of the user based on the audio analysis. At block 1304, processing logic examines the user dimensions to determine whether a first predetermined condition has been satisfied. At block 1305, processing logic updates a notification attribute of the user profile of the user. At block 1306, processing logic invokes an escalation process when a second predetermined condition is satisfied.

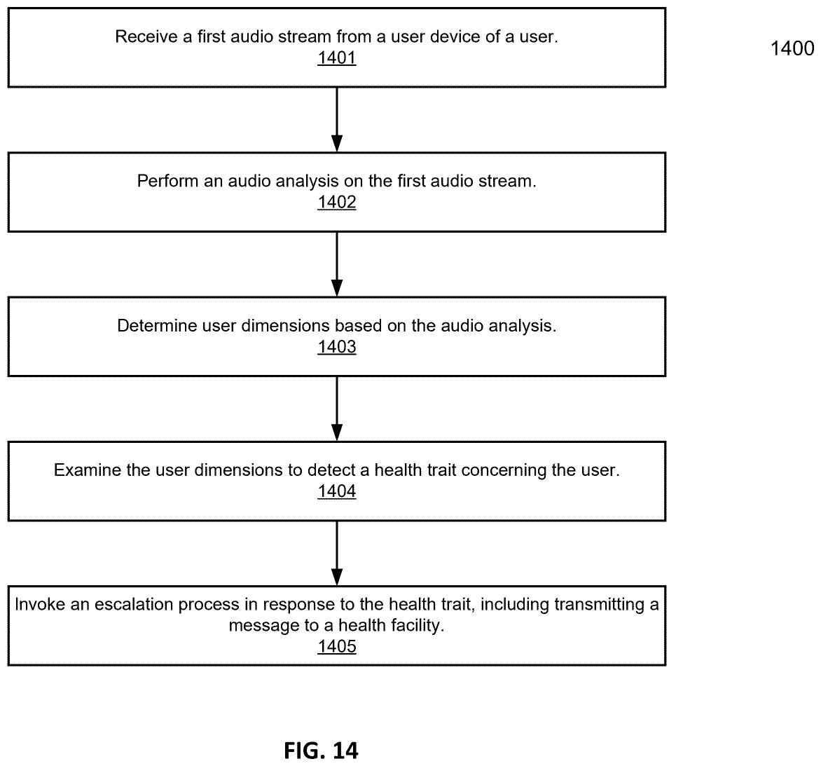

FIG. 14 is a flow diagram illustrating a process of voice paring services according to one embodiment. Process 1400 may be performed by processing logic, which may include software, hardware, or a combination thereof. For example, process 1400 may be performed by a digital agent, a server, and/or a contact center. Referring to FIG. 14, at block 1401, processing logic receives a first audio stream from a user device of a user. At block 1402, processing logic performs an audio analysis on the first audio stream. At block 1403, processing logic determines user dimensions based on the audio analysis. At block 1404, processing logic examines the user dimensions to detect a health trait concerning the user. At block 1405, processing logic invokes an escalation process in response to the health trait, including transmitting a message to a health facility.

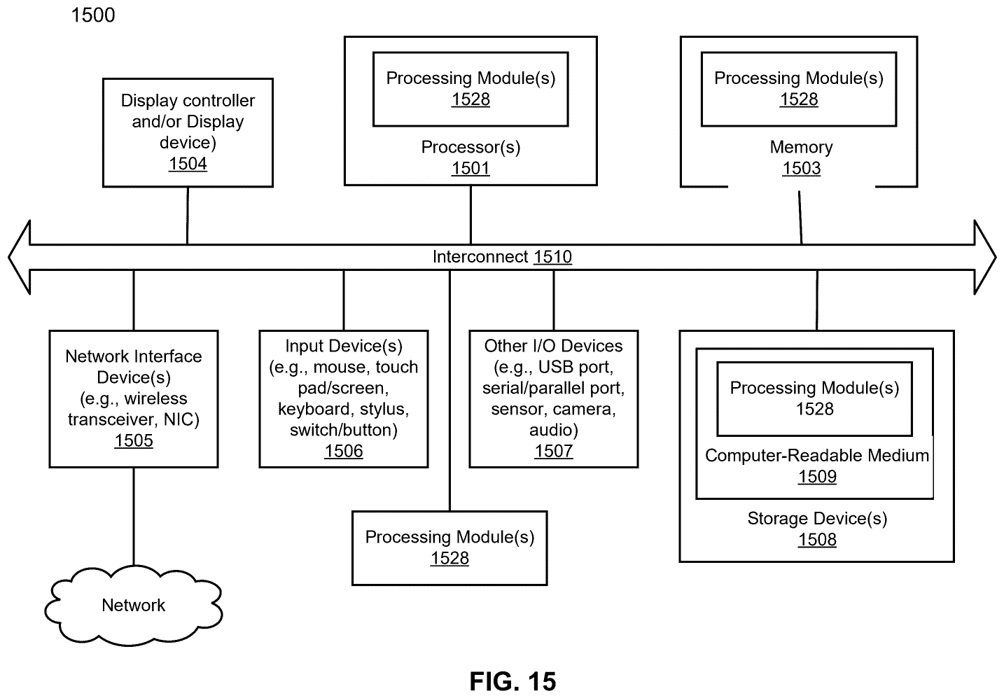

FIG. 15 is a block diagram illustrating an example of a data processing system which may be used with one embodiment of the invention. For example, system 1500 may represent any of data processing systems described above performing any of the processes or methods described above, such as, for example, a client device or a server described above, such as, for example, user devices 101, server 103, or systems 105-107, as described above.

System 1500 can include many different components. These components can be implemented as integrated circuits (ICs), portions thereof, discrete electronic devices, or other modules adapted to a circuit board such as a motherboard or add-in card of the computer system, or as components otherwise incorporated within a chassis of the computer system.

Note also that system 1500 is intended to show a high level view of many components of the computer system. However, it is to be understood that additional components may be present in certain implementations and furthermore, different arrangement of the components shown may occur in other implementations. System 1500 may represent a desktop, a laptop, a tablet, a server, a mobile phone, a media player, a personal digital assistant (PDA), a Smartwatch, a personal communicator, a gaming device, a network router or hub, a wireless access point (AP) or repeater, a set-top box, or a combination thereof. Further, while only a single machine or system is illustrated, the term “machine” or “system” shall also be taken to include any collection of machines or systems that individually or jointly execute a set (or multiple sets) of instructions to perform any one or more of the methodologies discussed herein.

In one embodiment, system 1500 includes processor 1501, memory 1503, and devices 1505-1508 via a bus or an interconnect 1510. Processor 1501 may represent a single processor or multiple processors with a single processor core or multiple processor cores included therein. Processor 1501 may represent one or more general-purpose processors such as a microprocessor, a central processing unit (CPU), or the like. More particularly, processor 1501 may be a complex instruction set computing (CISC) microprocessor, reduced instruction set computing (RISC) microprocessor, very long instruction word (VLIW) microprocessor, or processor implementing other instruction sets, or processors implementing a combination of instruction sets. Processor 1501 may also be one or more special-purpose processors such as an application specific integrated circuit (ASIC), a cellular or baseband processor, a field programmable gate array (FPGA), a digital signal processor (DSP), a network processor, a graphics processing unit (GPU), a network processor, a communications processor, a cryptographic processor, a co-processor, an embedded processor, or any other type of logic capable of processing instructions.

Processor 1501, which may be a low power multi-core processor socket such as an ultra-low voltage processor, may act as a main processing unit and central hub for communication with the various components of the system. Such processor can be implemented as a system on chip (SoC). Processor 1501 is configured to execute instructions for performing the operations and steps discussed herein. System 1500 may further include a graphics interface that communicates with optional graphics subsystem 1504, which may include a display controller, a graphics processor, and/or a display device.

Processor 1501 may communicate with memory 1503, which in one embodiment can be implemented via multiple memory devices to provide for a given amount of system memory. Memory 1503 may include one or more volatile storage (or memory) devices such as random access memory (RAM), dynamic RAM (DRAM), synchronous DRAM (SDRAM), static RAM (SRAM), or other types of storage devices. Memory 1503 may store information including sequences of instructions that are executed by processor 1501, or any other device. For example, executable code and/or data of a variety of operating systems, device drivers, firmware (e.g., input output basic system or BIOS), and/or applications can be loaded in memory 1503 and executed by processor 1501. An operating system can be any kind of operating systems, such as, for example, Windows® operating system from Microsoft®, Mac OS®/iOS® from Apple, or Android® from Google.

System 1500 may further include IO devices such as devices 1505-1508, including network interface device(s) 1505, optional input device(s) 1506, and other optional IO device(s) 1507. Network interface device 1505 may include a wireless transceiver and/or a network interface card (NIC). The wireless transceiver may be a WiFi transceiver, an infrared transceiver, a Bluetooth transceiver, a WiMax transceiver, a wireless cellular telephony transceiver, a satellite transceiver (e.g., a global positioning system (GPS) transceiver), or other radio frequency (RF) transceivers, or a combination thereof. The NIC may be an Ethernet card.

Input device(s) 1506 may include a mouse, a touch pad, a touch sensitive screen (which may be integrated with display device 1504), a pointer device such as a stylus, and/or a keyboard (e.g., physical keyboard or a virtual keyboard displayed as part of a touch sensitive screen). For example, input device 1506 may include a touch screen controller coupled to a touch screen. The touch screen and touch screen controller can, for example, detect contact and movement or break thereof using any of a plurality of touch sensitivity technologies, including but not limited to capacitive, resistive, infrared, and surface acoustic wave technologies, as well as other proximity sensor arrays or other elements for determining one or more points of contact with the touch screen.

IO devices 1507 may include an audio device. An audio device may include a speaker and/or a microphone to facilitate voice-enabled functions, such as voice recognition, voice replication, digital recording, and/or telephony functions. Other IO devices 1507 may further include universal serial bus (USB) port(s), parallel port(s), serial port(s), a printer, a network interface, a bus bridge (e.g., a PCI-PCI bridge), sensor(s) (e.g., a motion sensor such as an accelerometer, gyroscope, a magnetometer, a light sensor, compass, a proximity sensor, etc.), or a combination thereof. Devices 1507 may further include an imaging processing subsystem (e.g., a camera), which may include an optical sensor, such as a charged coupled device (CCD) or a complementary metal-oxide semiconductor (CMOS) optical sensor, utilized to facilitate camera functions, such as recording photographs and video clips. Certain sensors may be coupled to interconnect 1510 via a sensor hub (not shown), while other devices such as a keyboard or thermal sensor may be controlled by an embedded controller (not shown), dependent upon the specific configuration or design of system 1500.

To provide for persistent storage of information such as data, applications, one or more operating systems and so forth, a mass storage (not shown) may also couple to processor 1501. In various embodiments, to enable a thinner and lighter system design as well as to improve system responsiveness, this mass storage may be implemented via a solid state device (SSD). However in other embodiments, the mass storage may primarily be implemented using a hard disk drive (HDD) with a smaller amount of SSD storage to act as a SSD cache to enable non-volatile storage of context state and other such information during power down events so that a fast power up can occur on re-initiation of system activities. Also a flash device may be coupled to processor 1501, e.g., via a serial peripheral interface (SPI). This flash device may provide for non-volatile storage of system software, including a basic input/output software (BIOS) as well as other firmware of the system.

Storage device 1508 may include computer-accessible storage medium 1509 (also known as a machine-readable storage medium or a computer-readable medium) on which is stored one or more sets of instructions or software (e.g., module, unit, and/or logic 1528) embodying any one or more of the methodologies or functions described herein. Processing module/unit/logic 1528 may represent any of the components described above, such as, for example, digital agents, audio analysis modules, or CLMs as described above. Processing module/unit/logic 1528 may also reside, completely or at least partially, within memory 1503 and/or within processor 1501 during execution thereof by data processing system 1500, memory 1503 and processor 1501 also constituting machine-accessible storage media. Processing module/unit/logic 1528 may further be transmitted or received over a network via network interface device 1505.

Computer-readable storage medium 1509 may also be used to store the some software functionalities described above persistently. While computer-readable storage medium 1509 is shown in an exemplary embodiment to be a single medium, the term “computer-readable storage medium” should be taken to include a single medium or multiple media (e.g., a centralized or distributed database, and/or associated caches and servers) that store the one or more sets of instructions. The terms “computer-readable storage medium” shall also be taken to include any medium that is capable of storing or encoding a set of instructions for execution by the machine and that cause the machine to perform any one or more of the methodologies of the present invention. The term “computer-readable storage medium” shall accordingly be taken to include, but not be limited to, solid-state memories, and optical and magnetic media, or any other non-transitory machine-readable medium.

Processing module/unit/logic 1528, components and other features described herein can be implemented as discrete hardware components or integrated in the functionality of hardware components such as ASICS, FPGAs, DSPs or similar devices. In addition, processing module/unit/logic 1528 can be implemented as firmware or functional circuitry within hardware devices. Further, processing module/unit/logic 1528 can be implemented in any combination hardware devices and software components.

Note that while system 1500 is illustrated with various components of a data processing system, it is not intended to represent any particular architecture or manner of interconnecting the components; as such details are not germane to embodiments of the present invention. It will also be appreciated that network computers, handheld computers, mobile phones, servers, and/or other data processing systems which have fewer components or perhaps more components may also be used with embodiments of the invention.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques shown in the figures can be implemented using code and data stored and executed on one or more electronic devices. Such electronic devices store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals-such as carrier waves, infrared signals, digital signals).

The processes or methods depicted in the preceding figures may be performed by processing logic that comprises hardware (e.g. circuitry, dedicated logic, etc.), firmware, software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

In the foregoing specification, embodiments of the invention have been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

What is claimed is:

1. A computer-implemented method for providing support services, the method comprising:
receiving, by a digital agent hosted at a server associated with a contact center over a network, a first audio stream from a user device of a user, the first audio stream spoken by the user to inquire about a product provided by a product provider as a first client of the contact center, wherein the contact center is configured to provide support services for a plurality of products provided by a plurality of clients via a plurality of communication channels, wherein each of the plurality of clients represents one of a product manufacturer, a product distributer, a product retailer, or a service provider of the product;
performing an audio analysis on the first audio stream to determine a plurality of dimensions associated with the user, the dimensions of the user including voice characteristics of the user, the voice characteristics including vocal pitch and speech patterns of the user;
performing a content analysis on content produced by the first audio stream, including:
invoking a custom language model (CLM) on the content of the first audio stream as an input, wherein the CLM was specifically trained and customized for the product provided by the product provider via machine learning, and
generating a response to the inquiry about the product based on an output of the CLM;
generating a second audio stream based on the response and the dimensions of the user, such that at least some of voice characteristics of the second audio stream are similar to the voice characteristics of the user; and
transmitting the second audio stream to the user device over the network.

2. The method of claim 1, further comprising storing the dimensions of the user in a user profile of the user in a storage device, wherein the dimensions of the user can be retrieved from the user profile and used to generate subsequent responses in response to subsequent inquiries from the user.

3. The method of claim 1, wherein determining dimensions of the user comprises:
determining a gender of the user based on the voice characteristics of user; and
determining an age of the user based on the voice characteristics of the user, wherein the second audio stream is generated with a voice spoken by a person with the same gender and similar age of the user.

4. The method of claim 1, wherein determining dimensions of the user comprises determining a native language spoken by the user based on the voice characteristics of the user, wherein the second audio stream is generated with a voice spoken by a person with an accent similar to the user.

5. The method of claim 1, wherein determining dimensions of the user comprises determining an intention of the user based on the first audio stream, and wherein prior to performing the content analysis, the method further comprises:
determining whether the intention of the user matches one of a plurality of predetermined intentions; and
in response to determining that the intention of the user matches one of the plurality of predetermined intentions, starting a processing flow corresponding to the matched intention to generate the response to the inquiry about the product.

6. The method of claim 5, wherein the processing flow is one of a plurality of processing flows associated with the plurality of intentions respectively, and the processing flow is started without invoking the CLM.

7. The method of claim 6, wherein the CLM is invoked to produce the response when the intention of the user does not match any of the plurality of predetermined intentions.

8. The method of claim 1, wherein performing an audio analysis comprises:
determining a dimension score for each of the dimensions of the user, wherein a dimension score represents a state of the corresponding dimension;
calculating a customer voice index (CVI) based on the dimension scores of the dimensions of the user using a predetermined algorithm; and
storing the CVI in the user profile of the user.

9. The method of claim 8, wherein each of the dimension scores is associated with a weight factor when calculating the CVI using the predetermined algorithm.

10. The method of claim 8, further comprising:
selecting a text-to-speech (TTS) module from a plurality of TTS modules based on the CVI, wherein each of the TTS modules is configured to generate a voice with different voice characteristics; and
invoking the selected TTS module to convert the response into the second audio stream.

11. The method of claim 8, further comprising selecting the CLM from a plurality of CLMs associated with the product based on the CVI.

12. The method of claim 1, further comprising converting the first audio stream into a text stream using a speech-to-text (STT) module, wherein the CLM is invoked on the text stream as the input to generate the response.

13. A non-transitory machine-readable medium having instructions stored therein, which when executed by a processor, cause the processor to perform a method for providing support services, the method comprising:
receiving, by a digital agent hosted at a server associated with a contact center over a network, a first audio stream from a user device of a user, the first audio stream spoken by the user to inquire about a product provided by a product provider as a first client of the contact center, wherein the contact center is configured to provide support services for a plurality of products provided by a plurality of clients via a plurality of communication channels, wherein each of the plurality of clients represents one of a product manufacturer, a product distributer, a product retailer, or a service provider of the product;
performing an audio analysis on the first audio stream to determine a plurality of dimensions associated with the user, the dimensions of the user including voice characteristics of the user, the voice characteristics including vocal pitch and speech patterns of the user;
performing a content analysis on content produced by the first audio stream, including:
invoking a custom language model (CLM) on the content of the first audio stream as an input, wherein the CLM was specifically trained and customized for the product provided by the product provider via machine learning, and
generating a response to the inquiry about the product based on an output of the CLM;
generating a second audio stream based on the response and the dimensions of the user, such that at least some of voice characteristics of the second audio stream are similar to the voice characteristics of the user; and
transmitting the second audio stream to the user device over the network.

14. The machine-readable medium of claim 13, wherein the method further comprises storing the dimensions of the user in a user profile of the user in a storage device, wherein the dimensions of the user can be retrieved from the user profile and used to generate subsequent responses in response to subsequent inquiries from the user.

15. The machine-readable medium of claim 13, wherein determining dimensions of the user comprises:
determining a gender of the user based on the voice characteristics of user; and
determining an age of the user based on the voice characteristics of the user, wherein the second audio stream is generated with a voice spoken by a person with the same gender and similar age of the user.

16. The machine-readable medium of claim 13, wherein determining dimensions of the user comprises determining a native language spoken by the user based on the voice characteristics of the user, wherein the second audio stream is generated with a voice spoken by a person with an accent similar to the user.

17. The machine-readable medium of claim 13, wherein determining dimensions of the user comprises determining an intention of the user based on the first audio stream, and wherein prior to performing the content analysis, the method further comprises:
determining whether the intention of the user matches one of a plurality of predetermined intentions; and
in response to determining that the intention of the user matches one of the plurality of predetermined intentions, starting a processing flow corresponding to the matched intention to generate the response to the inquiry about the product.

18. The machine-readable medium of claim 17, wherein the processing flow is one of a plurality of processing flows associated with the plurality of intentions respectively, and the processing flow is started without invoking the CLM.

19. The machine-readable medium of claim 18, wherein the CLM is invoked to produce the response when the intention of the user does not match any of the plurality of predetermined intentions.

20. A data processing system operating as a server, comprising:
a processor; and
a memory having instructions stored therein, which when executed by the processor, cause the processor to perform a method for providing support services, the method comprising:
receiving, by a digital agent hosted at the server associated with a contact center over a network, a first audio stream from a user device of a user, the first audio stream spoken by the user to inquire about a product provided by a product provider as a first client of the contact center, wherein the contact center is configured to provide support services for a plurality of products provided by a plurality of clients via a plurality of communication channels, wherein each of the plurality of clients represents one of a product manufacturer, a product distributer, a product retailer, or a service provider of the product,
performing an audio analysis on the first audio stream to determine a plurality of dimensions associated with the user, the dimensions of the user including voice characteristics of the user, the voice characteristics including vocal pitch and speech patterns of the user,
performing a content analysis on content produced by the first audio stream, including:
invoking a custom language model (CLM) on the content of the first audio stream as an input, wherein the CLM was specifically trained and customized for the product provided by the product provider via machine learning, and
generating a response to the inquiry about the product based on an output of the CLM,
generating a second audio stream based on the response and the dimensions of the user, such that at least some of voice characteristics of the second audio stream are similar to the voice characteristics of the user, and
transmitting the second audio stream to the user device over the network.
